(19) United States
(12) Patent Application Publication
(10) Pub. No.: US 2021/0213080 A1
(43) Pub. Date: Jul. 15, 2021

(54) Specialty Pharmaceutically Active Agent Composites

(71) Applicant: Jordan Seth Rubin, College Grove, TN (US)

(72) Inventor: Jordan Seth Rubin, College Grove, TN (US)

(21) Appl. No.: 17/144,452

(22) Filed: Jan. 8, 2021

Related U.S. Application Data
(60) Provisional application No. 62/959,311, filed on Jan. 10, 2020.

(51) Int. Cl. A61K 36/18

(52) U.S. Cl. CPC A61K 36/18

(57) ABSTRACT

The present invention is a pharmaceutical cocktail of at least two botanical leaves (or extracts therefrom) specifically harvested from trees or plants that bear edible fruit, and including no other leaves of any kind. The combination of leaves from multiple families or at the very least genera provides the greatest variety of phytochemicals. A prime nonlimiting example is a combination of leaves such as Soursop (Annonaceae Family, Annona Genus), Blackberry (Rosaceae Family, Rubus Genus), Lemon (Rutaceae Family, Citrus Genus). In an embodiment of the invention, at least twelve leaf types (from at least different species if not higher biological categories) are admixed into such an active agent admixture—again, with each leaf type coming from a plant or tree that bears edible fruit. The present compositions and extracts always contain a minimum of 25 distinct active phytochemicals, which together provide nutraceutical and pharmaceutical action as to animals and humans.

### FIELD OF THE INVENTION

Medicament composites which are drawn from heretofore unappreciated botanical classification(s) of plant harvest or extraction sources, to prepare broad spectrum treating medicaments as described herein.

### BACKGROUND OF THE INVENTION

At this writing, the world is very focused on personalized medicine, and in many cases this has been a boon for everyone, from patients to practitioners. Personalized assessments including advanced genetic and biochemical assessments are so critical, to current medical practice, that it is hard to imagine how anyone ever treated a patient without them. Moreover, the spoken or unspoken treatment mantra for any given condition or indication is typically now a desire for a “magic bullet,” that will treat the indication effectively but work no harm or other action elsewhere. All of this is a wonderful legacy of recent medical and pharmaceutical innovations, and nothing that follows is intended to negate such a positive milieu in any way.

At the other end of the innovation spectrum, at least in terms of history, are the pharmacy initiatives that measure their tenure in millenia, not decades. One such field is that of herbal medicine as it is traditionally practiced in, as a single example, the Peoples Republic of China. For ages and still today, many traditional pharmacies in China dispense herbs—and herbs only. The route of administration of these herbs is and was generally to brew them into so-called “teas,” designed for oral administration. The herbs themselves were and are highly prized and often showcased in expensive vitrines in the pharmacy showrooms, just as one would display fine jewelry. These pharmacies are a testament to ancient wisdom that understands how to provide pharmaceutically active agents from botanical sources, in a pharmaceopoeia that is well established and documented for medicament type and indication.

Some of the disadvantages with both traditional herbal medicine and modern targeted therapies include, without limitation, an excessive specialization as to a given indication, and a “look-down-the-wrong-end-of-a-telescope” mindset that emphasizes the particular rather than the big picture. In other words, ironically, even well-established herbal medicine traditions can sometimes fall into the same overspecialization trap that allopathic pharmaceuticals also can, failing to consider or to treat the whole person instead of a single or limited indication. In parallel with such disadvantages of hyper-specialization, huge populations of individuals throughout the world at this writing are not yet—socioeconomically or otherwise—in a position to benefit from multiple specialized pharmaceutical interventions, whether herbal or technology based. There are huge populations outside of the developed nations in which access to effective and affordable health care is still rare if nonexistent. Given the need for widely effective, inexpensive, effective, and time-tested medicaments throughout the world, to populations in increasingly dire need, it is regrettable that (prior to the present invention) no readily-available innovative medicament composite solutions were known or existed with truly broad applicability, rather than specialized focus. Therefore, despite millenia of scholarly and documented development in botanically-harvested or -derived pharmaceutically active agents, a need remains for a new botanical active agent specialty composite—not to mention a novel approach to innovating the same—that defines a new paradigm in harnessing the power of specialty composite botanicals.

### SUMMARY OF THE INVENTION

In order to meet this need, the present invention is a pharmaceutical cocktail of at least two botanical leaves specifically harvested from trees or plants that bear edible fruit, and including no other leaves of any kind. The combination of leaves from multiple families or at the very least genera provides the greatest variety of phytochemicals. A prime example is a combination of leaves such as Soursop (Annonaceae Family, Annona Genus), Blackberry (Rosaceae Family, Rubus Genus), Lemon (Rutaceae Family, Citrus Genus). In another embodiment of the invention, at least twelve leaf types (from at least different species if not higher biological categories) are admixed into such an active agent admixture—again, with each leaf type coming from a plant or tree that bears edible fruit and including no other type of leaf (that is, including no leaves from any trees or plants that do not bear edible fruit). Each leaf type must come, at a minimum, from a distinct genus and species of plant or tree bearing edible fruit. In many embodiments of the invention, the aggregated leaf types are extracted or activated with heating, steaming, fermentation, carbon dioxide extraction, or all four—to denature unwanted constituents and to render more bioavailable the desired components. Such aggregate specialty composition provide broad spectrum medicaments from natural sources that in turn create widely useful active agent compositions—particularly for under served populations in need of health care and medical intervention.

### DETAILED DESCRIPTION OF THE INVENTION

The present invention is a pharmaceutical cocktail of at least two botanical leaves specifically harvested from trees or plants that bear edible fruit, and including no other leaves of any kind. Personal experience and recently documented positive clinical outcomes by the inventor suggests that there is a synergistic effect upon combining myriad phytochemicals found within the leaves of genetically diverse plant families, genera and species. The combination of leaves from multiple families or at the very least genera provides the greatest variety of phytochemicals. A prime example is a combination of leaves such as Soursop (Annonaceae Family, Annona Genus), Blackberry (Rosaceae Family, Rubus Genus), Lemon (Rutaceae Family, Citrus Genus). In another embodiment of the invention, at least twelve leaf types are admixed into such an active agent admixture—again, with each leaf type coming from a plant or tree that bears edible fruit and including no other type of leaf (that is, including no leaves from any trees or plants that do not bear edible fruit). Each leaf type must come from a distinct genus and species of plant or tree bearing edible fruit. In many embodiments of the invention, the aggregated leaf types are extracted or activated with heating, steaming, fermentation, carbon dioxide extraction, or all four—to denature unwanted constituents and to render more bioavailable the desired components. As a best practice, the leaves are harvested before or just after the tree or plant sets fruit and in any case well before any fruit on the tree or plant ripens or fully matures. Medically active agents in the plants are enriched in the leaves—but only until the fruit sets and starts to ripen, when those medically active agents make their way to the fruits as part of the ripening process, and the leaves become relatively depleted of all their desirable constituents around “ripening time.” The present innovation is harnessing the broad spectrum medicament nature of two or more of these specific leaves from edible fruit bearing plants or trees, and not in any other combinations of leaves or other herbal remedies heretofore known. It will be immediately apparent that by using multiple leaves, the botanical composite is not generally “personalized medicine” but is, as to each combination, a “broad spectrum” medicament that contains a number of active agents and—the more diverse the leaf species choices, the broader the spectrum of constituents therein. This botanical and chemical diversity is deliberate, and the broad spectrum paradigm deliberately avoids the “targeted therapy” paradigm in favor of innovative combinations that have multiple uses in a variety of medical indications. Two to sixteen types of leaves may be combined, more particularly three to fifteen, four to fourteen, or five to thirteen leaf types. The inventor does not in any way intend to suggest that individual leaves of trees or plants that produce only edible fruit have not singly been used as pharmaceutical herbs at any time in history. The essence of the present invention is the harnessing of the power of multiple leaves, specifically from edible-fruit-bearing trees or plants and with leaf harvest prior to fruit ripening, to create a new class of specialty botanical composite active agent compositions. The fruit tree leaves are at least 14 days old but are harvested before the corresponding fruit is fully ripe. The optimal harvest window would take place between the time when the leaves are six weeks old and prior to the setting of fruit. This latter timeframe yields the highest concentration of bio-active phytochemicals. The present compositions are suitable for administration to both humans and animals. An example is the oak leaf, where research shows the highest antioxidant capacity is found in leaves harvested between May and July prior to acorns coming on. This represents a range of 6-12 weeks, varying by climate.

When the leaves for inclusion in the present composites are harvested, they should be collected through normal pruning of the trees or plants in question. This invention is meant to implement a sustainable pharmacopoeia, especially in third world nations where it is most needed, and leaf stripping or irresponsible harvesting of such plants or trees would be anathema to this goal.

With multiple, diverse leaves, one harnesses the power of admixed botanicals to create a truly broad spectrum medicament—which can be used to treat intrinsic maladies, cancers or regulatory/modulatory disorders, even all at once! Thinking such as this requires breaking some mental taboos against medicines' having to be specific and targeted because, the truth is, they do not. When broad spectrum herbal composites are administered to a human in need of treatment, in general the human body itself can react beneficially to the subset of the broad spectrum that it needs (and the body knows better than the doctor)—and the rest “will do no harm.” In other words, the present herbal composites take the best (newly identified) subset of traditional herbal ingredients to make the most versatile, ubiquitously applicable health and wellness cocktail ever admixed to date. As a single non-limiting example, chlorogenic acid in blueberries is already known to block absorption of carbohydrates, which in turn treats both blood sugar regulation in diabetes as well as deterring cancer growth by restricting or halting cancer cells' access to sugar. The point is not to reinvent the administration of chlorogenic acid but to recognize that a 12 (or 2-16, 3-15, 4-14, or 5-13) leaf combination heated/fermented/carbon dioxide extracted heated infusion will include multiple beneficial constituents such as chlorogenic acid and many many other components, and the human body is thus capable of benefiting from the intervention necessary depending on current indication. So, while the great body of botanical pharmacology emphasized individual medicinal effects of each herb, the present composites celebrate the additive or synergistic effects of multiple specified leaves in beneficial combination.

Although exemplary only, a paradigmatic formulation of the present invention is, “Twelve Leaves,” that is, twelve leaf types (although see above nested number ranges for the scope of what applicant means by “multiple”) from trees or plants that bear edible fruit,. Ideally, there is as much genetic and other diversity among the leaves chosen as is possible, to create the broadest tenable spectrum of infusion or extract constituents. This broad spectrum is especially important when one acknowledges that while not all botanical constituents are intrinsically benign, they are typically present in amounts or molecular configurations that are beneficial even though closely related compounds at higher dosages would be deadly. (Anthocyanins can be very beneficial medicinal botanicals whereas, of course, cyanides can be lethal, as one of many examples). Because the present infusion is denatured through fermentation, heating, or extraction via ethanol, it includes both a broad spectrum of botanical active agents AND contains those multiple agents at relatively low individual concentrations, the presence of compounds or compositions that would be toxic at higher concentrations is optimized in that—in low amounts—such actives can provide a therapeutic effect nonetheless or, at worst, will do nothing (no harm).

To repeat, heating or fermentation of leaves will generally destroy or denature any antinutrients present in the leaves, which is why the optimal dosage form is a heated infusion (like a tea, but not a real tea, in the sense that the disclosed leaves are not true tea leaves). Generally speaking, this technology does not optimally embrace the unaltered extracts of multiple fruit tea leaves in combination—because too many unwanted compounds and constituents would be present. When applying various processing methods ie fermentation or heat treatment, to a combination of fruit trees leaves, certain naturally occurring compounds that may cause deleterious effects on the body are either transformed into innocuous or even beneficial substances or removed altogether. For example uncooked berries, leaves, bark and roots of the elderberry plant contain the chemicals lectin and cyanide, which can cause nausea, vomiting and diarrhea. Heating or fermenting the berries, leaves, bark and seeds will remove the cyanide. Thus, the heretofore largely overlooked leaves (not tea leaves) of trees or plants that produce edible fruit are the leaves used in the present multi-leaf combinations. By “tree that bears edible fruit” does not mean that the fruit in question has to be consumer friendly or delicious fruit, just edible—so coffee, oak, etc. are included in the list. Interestingly, green, white and black tea, and most herbs and spices in common culinary and medicinal use (echinacea, lavender and so forth) do NOT form examples of botanicals for use in the present infusions or extracts. The major list of perennial plants and trees that bear edible fruit and have leaves is well known in the art.

In all of the above disclosure, “trees” is to be understood to embrace perennial plants, which is to say, trees, bushes, hedges, shrubs and vines. In referring to “plants” both perennial and annual botanical organisms are included.

Trees (perennial trees, bushes, hedges, shrubs and vines (all perennials)) that may be used as sources for the present leaves including—without limitation—the following examples; Soursop; Mountain Soursop; Mango; Baobob; Lemon; Kaffir Lime; Lime; Sea Grape; Banana; Pink Banana; Grapefruit; Orange; Bergamot; Passionfruit; Passionfruit (purple); Pawpaw; Star Fruit; Pineapple; Date; Bitter Melon; Star Apple; Naranjilla; Jaboticaba; Noni; Wax Jambo; Breadfruit; Pomelo; Jaltamato; Yerba Mate; Quince; Gingko biloba; Plum; Natal Plum; June Plum; Camboge; Coffee; Indian Coffee Plum; Jackfruit; Blackberry; Blueberry; Sumac; Bilberry; Kiwi; Lychee; Strawberry; Raspberry; Persimmon; Cathay Persimmon; Boysenberry; Salmonberry; Cherry; Indian Cherry; Sweet Cherry; Mangosteen; Castana; Durian; Pomegranate; Cranberry; Grape; Muscadine; Olive; Wild Olive; Guava (Thai, Mexican and Pink); Neem; Turmeric; Herbert River Cherry; Lingonberry; Acai; Aronia; Tako; Fig (Fig (sacred)); Malaysian Ginseng; Mulberry; White Mulberry; Chinese Mulberry; Hawthorn; Moringa; Papaya; Goji; Pineapple; Currant (Black and Red); Sierra Currant; Avocado; Schisandra; Sea Buckthorn; Service Berry; Peach; Apple; Loguat; Coconut; Cacao; Walnut; Black Walnut; Pistachio; Galangal; Honeysuckle; Blue Honeysuckle; Garden Ginger; Red Torch Ginger; Cardamom; Oak; Ginkgo; Hazelnut; Chinese Hazelnut; Eleutherococcus (Siberian Ginseng); Almond; Cashew; Linden; Saw Palmetto; Beech; Katuk; Ash; Maple; Joshua Tree; Blue Sausage Fruit; Pine; Hibiscus (Abelmoschus manihot); Rose; Myrtle; Juniper; Inca Peanuts; Sacha Peanut; Mandrake; Cinnamon; Chocolate Vine; Clove; Nutmeg; Allspice; Brazilian Pepper Tree; Peruvian Pepper Tree; Long Pepper; Bayberry; Sapphire Berry; Sugar Apple; Tamarind; Sapodilla; Miracle Fruit; Cashew Apple-Anacardium occidentale; Ambarella-Spondias cytherea; Red Mombin-Spondias purpurea; Amra-Spondias pinnata; Bignay-Antidesma bunius; Grosella-Phyllanthus acidus; Balsam Pear; Garden Huckleberry; Chichiquelite Huckleberry; Smooth Chaste Tree; Lumbush, Popolo; Solanaceae macrocarpon; Solanum aethiopicum; Goldenberry-Physalis peruviana; Physalis angulata; Pterocarpus santalinoides; Bridelia micrantha; Vanilla; Camu Camu; Jujube; Cherimoya; Schwartzbeeren, Wonderberry, Lucuma; Key Lime; Astragalus; Ashwagandha; Wild Annona; Malayan Annona; Annona duckei; Annona paludosa; Sacha Inchi; Hops; Hackberry; Nannyberry; Medlar; Feijoas; Tamarillo; Andean Raspberry; Atherton Raspberry; Mao Luong; Rose of Sharon; Mysore Raspberry; Shrubby Fuchsia; Soncoya; Carob; Mesquite; Amla; Wild Grape; Mountain Grape; Oregon Grape; Assam Apple; Gac Fruit; Star Anise; and Sangre De Drago.

Two or more of the leaves of the above or other edible-fruit bearing trees or plants may be combined into the present invention formulations for oral or topical administration. In particular, without limitation, possible preparations include cold beverages, cold fermented beverages, inclusion in raw foods such as salads and smoothies; inclusion in fermented foods such as sauerkraut or kim chi; cooked vegetable preparations such as leafy greens are typically prepared; brewed “tea” like beverages brewed from loose or bagged leaves; dried fermented leaves as a dietary supplement taken alone or included in other foods or beverages; hot water extracts (concentrated liquids); hot water extracts (dried extracts); ethanol extracts (tincture); hydroethanolic extracts, carbon dioxide extracts and spent biomass/pomace. Such compositions may be administered by the oral or topical routes of administration to animals or humans in need of the constituent cocktails of such botanical constructs.

Without intending to be bound by the following theory, the mechanism of action of the present specialty botanicals is believed to include insulin reduction to within a normal range, infection amelioration and anti-inflammatory effect. The reason all three of these mechanisms is asserted for any combination of two or more leaves from edible fruit bearing organisms as described herein is that such leaves ubiquitously contain (when two or more are combined) constituents that are active in all of these categories. As result of these mechanisms of action, users can legitimately expect improvemet in longevity, cancer treatment, cancer survival, weight loss, diabetes management, memory preservation and cognitive function husbandry. Additional indications for the present active agent cocktails include (without limitation) Depression; Alzheimer's Disease; Dementia; Hyperlipidemia (high cholesterol); Fungal Infections (systemic); Bacterial Infections (systemic); Viral Infections (systemic); Diarrhea; Constipation; Acne; Eczema; Psoriasis; Ulcers; Kidney Stones; High Blood Pressure; Cardiovascular Disease (Atherosclerosis, Arteriosclerosis); Back Pain; Epilepsy; Asthma; Headache; Arthritis; Autoimmune Diseases; Cough; Common Cold; Flu; Menopause Symptoms; PMS; Gout; Nausea; Insomnia; Anemia; Prostatitis; Cystitis; Gum Disease; Menopausal Symptoms; Sports Performance; and Sports Recovery.

The invention is further illustrated in the following non-limiting example.

### EXAMPLE 1

Fill a large pot with 2.5 quarts or liters of pure water. Choose 12 leaves (single leaves or multiple leaves) from each of twelve trees or plants that bear edible fruit, such as (without limitation) Soursop; Mango; Baobob; Lemon; Kaffir Lime; Lime; Sea Grape; Banana; Grapefruit; Orange; Bergamot; Passionfruit; Pawpaw; Star Fruit; Pineapple; Date; Jaboticaba; Vitex; Eggfruit; Atemoya; Blue Grape; Prickly Pear; Jerusalem Thorn; Noni; Breadfruit; Quince; Plum; Coffee; Hikan; Jackfruit; Blackberry; Blueberry; Bilberry; Kiwi; Strawberry; Golden Alexandria Strawberry; Cape Gooseberry; Baobob; Loquat; Longan; Jabuticaba; Dragon Fruit (yellow, white, red); Dragon Tree; Mamey (Sapote); Black Sapote (Chocolate Pudding Fruit); White Sapote; Bearberry; Yumberry; Raspberry; Maqui Berry; Andea Raspberry; Persimmon; Boysenberry; Cherry; Mangosteen; Durian; Pomegranate; Cranberry; Grape; Muscadine; Olive; Guava; Lingonberry; Aronia; Fig; Mulberry; Hawthorn; Moringa; Papaya; Black Goji Berry; Pineapple; Currant (Black and Red); Avocado; Schisandra; Sea Buckthorn; Service Berry; Peach; Apple; Loguat; Coconut; Cacao; Walnut; Pistachio; Oak; Ginkgo; Hazelnut; Almond; Cashew; Linden; Saw Palmetto; Beech; Katuk; Ash; Maple; Joshua Tree; Appleberry; Pine; Hibiscus (Abelmoschus manihot); Rose; Myrtle; Juniper; Inca Peanuts; Mandrake; Cinnamon; Clove; Nutmeg; Allspice; Brazilian Pepper Tree; Peruvian Pepper Tree; Bayberry; Tamarind; Sapodilla; Cashew Apple-Anacardium occidentale; Ambarella-Spondias cytherea; Red Mombin-Spondias purpurea; Amra-Spondias pinnata; Bignay-Antidesma bunius; Grosella-Phyllanthus acidus; Balsam Pear; Garden Huckleberry; Lumbush, popolo; Solanaceae macrocarpon; Solanum aethiopicum; Goldenberry-Physalis peruviana; Physalis angulata; Pterocarpus santalinoides; Bridelia micrantha; Vanilla; Camu Camu; Cherimoya; Lucuma; Sacha Inchi; Hops; Hackberry; Nannyberry; Medlar; Feijoas; Tamarillo; and Sangre De Drago. Add 3 tablespoons of fresh leaves or 1 tablespoon of dried leaves (break leaves into small pieces) of each of the twelve chosen leaf types to the pot of water. Bring to a boil and simmer for 12 minutes. Remove from heat and cover for 20 minutes. Strain into large bowl. Pour into two-one quart/liter jars. The best practice is to make six to eight batches at one time. For treatment, each patient should consume one quart/liter per day, unsweetened. If several batches are made in the same day, leaves may be re-used as long as additional leaves are added to the re-used leaves.

### EXAMPLE 2

Below is a list of phytochemicals by fruit tree leaf, which are beneficial compounds (primarily antioxidants) found in a blend of 12 fruit tree leaves with each species belonging to a distinct biological family. Personal experience and recently documented positive clinical outcomes by the inventor suggest that there is a synergistic effect upon combining myriad phytochemicals found within the perennial fruit tree leaves of genetically diverse plant families, genera and species. Please note that the below list represents one embodiment of the invention only, for illustration, and recall that in all cases the reference is to the leaves of the tree or plant, not the fruit thereof. Even though the below exemplary list illustrates how there can easily be hundreds of active phytochemicals in the compositions of the present invention, at a minimum there will always be at least 25 active phytochemicals in the inventive composition—whether 25 phytochemicals come from at least two fruit tree leaf types, twelve fruit tree types, or more. By “active” is meant—biologically active phytochemicals as to a human or animal to which such phytochemicals are administered. It is therefore easily within the bounds of the present invention for the composition, or extract, to have 25 phytochemicals, 48 phytochemicals, 100 phytochemicals, 250 phytochemicals, or 350 phytochemicals identifiable therein.

### Soursop (Annonaceae Annona Muriatica)

Over 200 compounds have been isolated in the plant including alkaloids, phenols, acetogenins
   s-cadinene
   a-muurolene
   andrographolide
   t-cadinol ledene oxide
   a-cardinol
   b-caryoohyllene
   3-Octadexyloxy propyl ester
   Octadecane
   Gitoxigenin
   D-limonene
   a-pinene

### Moringa (Moringaceae Moringa Oleifera)

Vitamins
Carotenoids
Polyphenols
Phenolic Acids
   Gallic Acid
   Chlorogenic Acid

### Caffeic Acid

Flavonoids
   Myrecytin
   Quercetin
   Kaempferol
Alkaloids
   N,a-L-rhamnopyranosyl vincosamide
   Phenylacetonitrile pyrrolemarumine
   4′-hydroxyphenylethanamide-a-L-rhamnopyranoside
Glucosinolates
Isothiocyanates
Tannins
Saponins

### Pomegranate (Lythraceae Punica Granatum)

Flavonoids and Anthocyanidins:
   delphinidin, cyaniding and pelargonidin
Ellagic Acid, Eligitannin, Hydrosoluble Tannins
Chemical Metabolites: 3,8-dihydroxy-6H-dibenzo[b, d]pyran-6-one (urolithin A, UA)
Caffeic acid phenethyl ester (CAPE)
catechin and epicatechin (epigallocatechin-3-gallate)
Minerals, potassium, nitrogen, calcium, phosphorus, magnesium, and sodium,
Complex Polysaccharides
organic acids: ascorbic acid, citric acid, and malic acid
Anthocyanins: delphinidin-3-glucoside, cyanidin-3-glucoside, delphinidin-3,5-diglucoside, cyanidin-3,5-diglucoside, pelargonidin-3,5-diglucoside, and pelargonidin-3-glucoside with delphinidin-3,5-diglucoside

### Jabuticaba (Myrtaceae Plinia Cauliflora)

Anthocyanins
depside, jaboticabin
3,3′-dimethylellagic acid-4-O-sulfate

### Tamarillo (Solanaceae Solanum Betaceum)

ellagic acid, rutin, catechin, epicatechin, kaempferol-3-rutinoside, isorhamnetin-3-rutinoside. Beta carotene, Anthocyanins, potassium, folate, niacin, thiamine, copper, magnesium, manganese, phosphorus, selenium and zinc.

### Key Lime (Rutaceae Citrus Aurantifolia)

apigenin, hesperetin, kaempferol, limonoids, quercetin, naringenin, nobiletin, and rutin

### Avocado (Lauraceae Persea Americana)

Peptone, b-galactoside, glycosylated abscisic acid, alkaloids, cellulose, polygalacto urease, polyuronoids, cytochrome P-450, and volatile oils

### Mango (Anacardiaceae Mangifera Indica)

mangiferin, rhamnetin, catechin, epicatechin, iriflophenone 3-C-β-D-glucoside, gallic acid and other phenolic and flavonoid compounds

### Baobab (Malvaceae Bombacoideae Adansonia Digitata)

procyanidins and flavonol glycosides, with tiliroside as the major constituent

### Fig (Moraceae Ficus Carica)

benzaldehyde and coumarins (i.e., angelicin, marmesin, psoralen, umbelliferone, and bergapten), phytosterols such as beta-sitosterol and taraxasterol; and furanocoumarins such as: psoralen and bergapten

### Loquat (Rosaceae Eriobotrya Japonica)

ursolic acid, maslinic acid, euscaphic acid, 3-epicorosolic acid, pomolic acid, colosolic acid and its methylated derivative

### Olive (Oleaceae Olea Europaea)

contains many different compounds, specifically biophenols, which are thought to give the extract its varied therapeutic properties. The most abundant biophenol is oleuropein, a secoiridoid composed from elenolic acid and hydroxytyrosol.

I claim:

1. A botanical medicament combination comprising at least two different leaves taken from a perennial plant or perennial tree that bears edible fruit, with harvest having been at a time prior to the full ripening of said edible fruit on said plant or tree, with said leaves being combined in a medicament form suitable for: brewing into a heated extract for oral administration; or concentration into a potent liquid extract; or drying into a powder; or drying into a powdered extract, any of which is further incorporated in foods, beverages, pharmaceuticals, medical foods, dietary supplement liquids, powders, capsules, tablets or chews suitable for administration to an animal or human in need of a broad spectrum herbal medicament.

2. The botanical composition according to claim 1, wherein said leaves are initially pretreated by heating, steeping in the sun, carbon dioxide extraction, fermentation or ethanol or other solvent extraction prior to rendering the combined leaves into a composite for subsequent brewing, evaporation or drying.

3. The botanical composition according to claim 1 as formulated into a topical solution for the use as an ingredient in transdermal skin or body care formulations.

4. The botanical composition according to claim 1 wherein said leaves are harvested between 14 days after initial leaf growth and before the fruit of the plant is fully ripe.

5. The botanical composition according to claim 1 wherein said leaves are harvested between six weeks from formation of the leaves and before the fruit of the plant sets.

6. The botanical composition according to claim 1 wherein said leaves are harvested from different species.

7. The botanical composition according to claim 1 wherein said leaves are harvested from different genera.

8. The botanical composition according to claim 1 wherein said leaves are harvested from different families.

9. The botanical composition according to claim 1 wherein said leaves are taken from between two to sixteen separate perennial species of plants or trees that bear edible fruit and wherein said composition contains at least 25 unique active phytochemicals in or derived from said leaves.

10. The composition according to claim 6 wherein said composition contain at least 48 unique active phytochemicals in or derived from said leaves.

11. The composition according to claim 6 wherein said composition contain at least 100 unique active phytochemicals in or derived from said leaves.

11. The composition according to claim 6 wherein said composition contain at least 250 unique active phytochemicals in or derived from said leaves.

12. The composition according to claim 6 wherein said composition contain at least 350 unique active phytochemicals in or derived from said leaves.

13. The botanical composition according to claim 6 wherein said composition contains a twelve leaf combination and at least 25 unique active phytochemicals in or derived from said leaves.

14. The botanical composition according to claim 6 wherein said composition contains a twelve leaf combination and at least 48 unique active phytochemicals in or derived from said leaves.

15. The botanical composition according to claim 6 wherein said composition contains a twelve leaf combination and at least 100 unique active phytochemicals in or derived from said leaves.

16. The botanical composition according to claim 6 wherein said composition contains a twelve leaf combination and at least 250 unique active phytochemicals in or derived from said leaves.

17. The botanical composition according to claim 6 wherein said composition contains a twelve leaf combination and at least 350 unique active phytochemicals in or derived from said leaves.
